(19) United States
(12) Patent Application Publication
(10) Pub. No.: US 2012/0089045 A1
(43) Pub. Date: Apr. 12, 2012

(54) MEASUREMENT SYSTEM FOR EVALUATING THE SWALLOWING PROCESS AND/OR FOR DETECTING ASPIRATION

(75) Inventors: Rainer Ottis Seidl, Berlin (DE); Holger Nahrstaedt, Berlin (DE); Thomas Schauer, Berlin (DE)

(73) Assignee: TECHNISCHE UNIVERSITAET BERLIN, Berlin (DE)

(21) Appl. No.: 13/257,035

(22) PCT Filed: Mar. 22, 2010
(86) PCT No.: PCT/DE2010/000329

§ 371 Date: Nov. 28, 2011

(30) Foreign Application Priority Data
Mar. 20, 2009 (DE) 10 2009 013 925.7
Jun. 29, 2009 (DE) 10 2009 031 564.0
Jul. 9, 2009 (DE) 10 2009 033 271.5
Sep. 24, 2009 (DE) 10 2009 042 964.6

(51) Int. Cl. A61B 5/053

(52) U.S. Cl. 600/547

(57) ABSTRACT

The invention relates to the use of measurement system for evaluating a swallowing process, preferably a closure of the airway during the swallowing process and/or an aspiration. The measurement system can be used for supporting therapy in case of swallowing disorders and/or for diagnosing changes in swallowing sequence.

### SUMMARY

The invention relates to the use of a measurement system for assessing a process of swallowing, preferably a closure of the airway during the swallowing process and/or an aspiration. The measurement system can be used for supporting therapy in cases of swallowing disorders and/or for diagnosing changes in the swallowing sequence.

### DESCRIPTION

Swallowing is one of the basic necessities of humans in order to stay alive. Disorders of this function, also referred to as dysphagia, can be fatal within a short period of time due to dehydration or starvation or as a result of secondary diseases such as pneumonia.

The incidence of dysphagia is high and mostly implies an acute threat to the life of affected patients. In the United States the percentage of patients suffering from swallowing disorders is about 14% in acute care hospitals and up to 50% in nursing homes. Aspiration pneumonia is the fourth leading cause of death among the over 65-year-old in the United States. With 25%, stroke represents the leading cause of all dysphagias. In England, 30,000 new patients with swallowing disorders following cerebral infarction are to be expected each year. However, both cerebral infarctions and cerebral hemorrhages can be concerned. Within two weeks after the disease event, 41% of the patients suffer from symptoms of dysphagia, and 16% during the chronic phase. The second leading cause of dysphagia is craniocerebral trauma. During the acute phase, a large proportion of patients are not capable of oral food intake. After one year of chronic stage or rehabilitation, swallowing disorders are mentioned in 10-14% of the cases (Winstein C J (1983); Yorkston K M, et al. (1989)).

Despite intensive efforts, there remains a high risk for patients with dysphagia, and more than 48% of aspiration pneumonias during one year following an acute cerebrovascular disease are reported in a paper by Johnson, McKenzie and Sievers (1993).

The central control of swallowing processes proceeds via swallowing centers in the brain stem (“pattern generators”). These processes are stimulated, on the one hand, by olfactory, gustatory and visual stimuli and, on the other hand, by the sensation of hunger, and modulated by higher suprabulbar centers. Thus, one or more pontine, one pontine-medullary and two bulbar swallowing centers have been postulated in the reticular formation, which are active already at birth. Essential to the success of swallowing is an intact interplay of the swallowing centers with motor and sensory cranial nerve core areas and cranial nerve fibers. A disorder of the suprabulbar centers with consecutive misinformation to the “pattern generators” leads to dysphagia.

Electromyographic measurements of the muscles in the pharyngeal-esophageal region show that a somatotopic representation exists, which exhibits a handedness-independent hemispheric difference and is asymmetric. Transfer to the muscles proceeds via five pairs of cranial nerves (trigeminal nerve V, facial nerve VII, glossopharyngeal nerve IX, vagus nerve X, hypoglossal nerve XII) and 3 cervical nerves which form the cervical plexus. They are required in order to ensure the necessary afferents and efferents of the swallowing process which proceeds in four or five phases.

The preparatory phase of swallowing is voluntarily controllable. The food is introduced, placed on the anterior/mid third of the tongue and examined by specific receptors with regard to smell, taste, temperature and volume. Solid and semi-solid foods are comminuted, mixed with saliva and formed into a bolus which is enclosed by the tongue in the anterior to mid palate area in the “tongue bowl” at the end of the chewing phase. The average bolus volume is 5-20 ml.

The complex pharyngeal phase begins with the triggering of the swallowing reflex and ends with the opening of the upper esophageal sphincter and takes 0.7 to 1 s. It is not voluntarily controllable. During this phase the pharyngeal space expands for bolus passage, pressure is built up to promote bolus transportation, and the airways are closed to protect against aspiration. Rapid piston-like movements of the tongue support passage of the bolus into the hypopharynx. Peristaltic movements of the pharyngeal wall support the piston function of the tongue. Depending on the bolus volume, hyoid bone and larynx move upwardly due to contraction of the suprahyoid muscles. This motion results in an expansion of space in the hypopharynx, positioning of the larynx under the root of the tongue to prevent aspiration, improved epiglottic tilting, and opening of the pharyngo-esophageal segment. To protect against aspiration, closure of the larynx proceeds in 3 stages: closure of the vocal folds, vertical approach of the adducted arytenoids to the base of the epiglottis, and epiglottic tilting to cover the laryngeal vestibule. Closure of the epiglottis is made possible by the bolus pressure from above, the downward muscular action of the aryepiglottic muscles, and the combined pressure as a result of the backward movement of the tongue and the laryngeal elevation. Opening of the upper esophageal sphincter is made possible by the anterior-superior movement of hyoid bone and larynx. The pharyngeal phase ends as soon as the bolus has reached the upper esophageal sphincter. Thereafter, the pharyngo-esophageal element, the tongue, hyoid and larynx return to their original positions. Velopharyngeal and laryngeal closures open up, and the pharyngo-esophageal element is closed.

The esophageal phase begins with the closure of the pharyngo-esophageal segment and lasts for 8 to 20 s. Bolus transportation proceeds by means of primary peristaltic waves induced by the swallowing reflex and, secondarily, by local stretch stimuli.

Today, different therapeutic approaches are utilized for dysphagias of different genesis. Apart from supportive measures such as dietary adaptation of the food consistency, surgical procedures such as tracheotomy or placement of a PEG are required in cases involving marked dysphagia to prevent or minimize complications. Further measures such as surgical closure of the larynx or laryngectomy fall outside the standard and are used in exceptional cases only.

Conservative dysphagia therapies can be roughly classified into two approaches. Sensory measures (cold, heat, taste, etc.) are intended to change the induction, coordination or extent of a swallowing process. To this end, the exterior and/or interior oral region is stimulated using sensory stimuli. By changing the body position, posture (turning the head), supportive movements (e.g. Shaker maneuver) or actions (e.g. Masako maneuver) during the swallowing process, motoric measures are intended to facilitate or allow passage of food through the pharynx into the esophagus and reduce or prevent aspiration. Preparation and support of such motor skills and motoric measures involve strengthening exercises dealing particularly with the movement of the tongue. Motoric procedures are used especially in isolated disorders, e.g. following surgery, and sensory measures are used in neurological diseases involving changes in perception. When dealing with complex neurological disorders where both sensory and motor components of the swallowing sequence are disturbed, complex therapeutic methods (F.O.T.T.) are being increasingly used today.

In addition to various clinical examination methods attempting to assess the risk of aspiration in standardized food intake, two examination methods for assessing the swallowing process are regarded as gold standard today.

Videofluoroscopy (VFS) is an X-ray examination of the swallowing process. The patient swallows foods including a contrast medium. The swallowing process is recorded as a video using fluoroscopy. The examination is documented in two planes (frontal and lateral). Slow motion resolution allows accurate assessment of the separate swallowing phases and possible interferences.

When using videofluoroscopy it is possible to observe the entire process of swallowing during the pharyngeal phase of swallowing. The disadvantages lie in the considerable technical complexity and the exposure to radiation during the examination. Automated assessment of images and analysis of results are not possible.

In fiberoptic endoscopic examination of swallowing (FEES), the swallow is examined using a transnasally introduced, flexible endoscope. What is observed and assessed is the ingestion of saliva and foods of varying consistency (e.g. colored water, green jello and bread). Compared to the VFS, the advantage of this method lies in lower exposure to radiation, so that examinations can be repeated, e.g. in documenting the course of a therapy. The disadvantage is that assessments cannot be made during the oral phase and during the intradeglutitive pharyngeal phase, because the camera image appears white (“white out”) as a result of an approach of tongue and posterior pharyngeal wall during actual swallowing. Automated recording of the swallowing process is not possible in all its phases.

Another measuring method is electromyography (EMG). In this method, the electrical activity on muscles and groups of muscles is detected. The method provides statements as to the onset and end of muscle activity and intensity of muscle contraction. The EMG is suitable for diagnosing neuromuscular diseases. At present, EMG is rarely used to assess swallowing disorders. This method so far has not found entrance into the diagnosis of the dynamic swallowing process.

Sonography allows real-time assessment of anatomical structures using ultrasound. However, sonography is of limited use for assessing swallowing disorders. Possible uses are observing the tongue function during the oral phase of swallowing and assessing the movements of hyoid bone and thyroid cartilage. However, processes in the pharyngeal space are difficult to detect due to the different types of tissue present therein.

A piezoelectric sensor can be used for detecting the upward and downward movements of the laryngeal skeleton during swallowing. To this end, the sensor is placed between the cricoid and thyroid cartilage on the midline of the neck. Firm skin contact must be provided by means of a patch. Changes in pressure resulting from the cricoid cartilage gliding beneath the sensor are sensed by the latter. As an alternative to a piezoelectric sensor, an accelerometer can be used to detect the movements of the larynx. The accelerometer is positioned in the same way as the above-described piezoelectric sensor.

In the method of neck auscultation, sounds caused by swallowing are detected on the neck using a microphone or stethoscope. At present, research is making attempts to diagnose swallowing disorders (e.g. aspiration) through acoustic analysis of the sounds. Furthermore, examinations relating to the coordination of breathing and swallowing, frequency of swallowing and detection of events in the swallowing process have been conducted using the auscultation method. Despite intensive research, only a few researchers so far have used this method because of the uncertain results.

Kob et al. have described a multi-channel electroglottographic method which can be used to detect the 2D position of the larynx with a frequency of 5 Hz. The system described is based on U.S. Pat. No. 4,909,261.

Electroglottography is primarily used to detect closure of the glottis. In a secondary aspect, the measurement can be used to detect movements of the larynx. Due to the placement of the electrodes on both sides of the larynx it is not possible to determine the movement of other structures involved in swallowing, such as the base of the tongue. Moreover, multi-channel electroglottography is very complex because of the large number of electrodes being used.

The group of Kusuhara, Yamamoto, Nakamura et al. has investigated the scope of four-electrode bioimpedance measurements of swallowing. The passive electrical properties of body tissue can be summarized as bioimpedance (BI). The BI is detected via the voltage drop caused by a constant amplitude sinusoidal current flow through the tissue.

The electrodes were placed on the sternocleidomastoid muscle and the larynx on both sides of the neck. A frequency of 50 kHz was used for BI measurement. The measuring method was referred to as Impedance Pharyngography (IP) by the authors. In an investigation by Yamamoto et al. the reproducibility of the measurement curve upon slight changes in the electrode positions was determined. It was found that the curves of impedance pharyngography had nearly identical profiles even after changing the electrode positions. The resulting measurement curve was interpreted to reflect the entire swallowing sequence (oral, pharyngeal, esophageal phases). Movements of larynx, pharynx, neck and esophagus are regarded as the cause of change in impedance. The impedance measurement described therein uses a grounded power source where current flow is regulated and controlled on only one of the two current electrodes. Such a method may give rise to two major problems:
(1) Touching the patient during measurement, e.g. touching by a therapist, may give rise to current flow through the therapist. As a consequence, part of the current from the controlled electrode will not flow through the patient, resulting in a drop in current amplitude on the non-controlled current electrode. As a result, the electric field in the measurement volume undergoes massive changes, which can lead to dramatic errors in bioimpedance measurement.
(2) Another problem with this method is that common-mode interferences (e.g. 50 Hz mains hum) cannot be suppressed completely due to different electrode-skin contact resistances on the two current electrodes. This may give rise to a direct current during bioimpedance measurement, so that undesirable tissue irritation as a result of electrolytic reaction on the electrodes may occur.

In addition, the measuring method described by the Japanese groups is not robust to low-frequency interferences not filtered out by the 25 Hz low pass being used. These include e.g. interferences generated by cable motion.

In collar impedance tomography, 16 electrodes are attached in a transverse plane on the neck at the level of the thyroid cartilage and the third cervical vertebra. The spatial resolution of the measurement is in the centimeter range. It was found that precise determination of bolus transit times is possible by using impedance tomography, wherein the variability of the measurements is substantially smaller for larger bolus volumes (20 ml).

In this study it has been hypothesized that the change of the measured signal, i.e. the decrease in electrical resistance, correlates with the decrease in the amount of air (curve volume) above the laryngeal vestibule. The curve volume was calculated after 20% amplitude (FW20) and 50% amplitude (FW50), revealing a positive correlation between the calculated parameters and the transit time determined in videofluoroscopy. In addition, the results suggested a connection with the maximum of the amplitude and the conductivity of the solution under investigation. It was found that the calculated results of measurement (FW20, FW50) were dependent on the bolus volume. This also applied to the reproducibility of the measured signal that increased with bolus volume. Age and gender also had an influence on the measurement result.

In impedance tomography a very large number of electrodes are taped to the patient's neck to observe the swallowing process. As a result of the multiplexing method being used, the time resolution (maximum frequency of 10 Hz) in this measuring method is poor.

The issue of aspiration is of crucial importance for the decision whether a patient may receive food or must be fed through a tube or even requires tracheotomy. Today, the VFS is regarded as gold standard when testing for aspiration. In many cases the FEES is used in everyday clinical practice. However, the latter allows only secondary conclusions about aspiration because the space below the vocal cords cannot be assessed without an existing tracheotomy.

The examinations outlined above may be performed only by physicians. In many cases, however, an assessment whether a patient should be fed orally is required in the daily practice of geriatric hospitals, nursing homes or rehabilitation facilities where the technical equipment does not permit such examinations and properly trained personnel is not available.

For safe food intake of a patient there is therefore an urgent need for a method allowing assessment of aspiration in a simple manner and without substantial technical effort and risk to the patient, even in patients having limited perception.

To protect the lungs from aspiration and pneumonia, closure of the larynx during the swallowing process is of crucial importance. At present, only VFS is available for a comprehensive survey of this process. Apart from the substantial technical efforts involved in VFS, the radiation exposure is a substantial drawback of this method. The FEES allows only secondary conclusions about the result of the swallowing process. Since both examination methods (VFS and FEES) can only be performed and evaluated by a physician, cost and effort required for such examinations are high. Automated examinations that could be performed by paramedical staff as well are not available at present. Diagnoses of swallowing disorders and exercises for improving the swallowing process are predominantly the domain of speech-language therapists, physiotherapists and ergotherapists. To date, there is no easy-to-use tool available that could be employed to test the success of a swallowing therapy using e.g. swallowing maneuvers. Also, it is not possible for patients to verify the success of their exercising efforts.

Automated detection of the swallowing processes is required to develop more advanced diagnostic procedures and improved training designs.

The technical object constituting the basis of the present invention was therefore to develop a measurement system which could be used for the assessment of the swallowing process and swallowing disorders such as aspiration, penetration or dysphagia and would not involve the disadvantages of the prior art. In particular, the measurement system should be suitable for frequent to permanent use.

Said object was accomplished by the independent claims, and advantageous embodiments can be inferred from the subclaims.

In a first embodiment the invention relates to the use of a measurement system comprising
two elements for applying a current,
the current being applied to the neck region, and a change in bioimpedance being detected
by two voltage measuring elements for
(i) detecting a closure of the airways during the swallowing process and/or
by two voltage measuring elements for
(ii) detecting the passage of non-gaseous substances through a cavity partially or completely surrounded by cartilage.

It was entirely surprising that the airway closure and the passage of non-gaseous substances through a cavity partially or completely surrounded by cartilage correlated with a measurable change in bioimpedance.

The measurement system can be used transcutaneously, subcutaneously, intratracheally and intraluminally. It can be utilized in the diagnosis, therapy and prevention of swallowing disorders and changes in the swallowing process, regardless of the underlying disease. The measurement system can also support the training of people in which the swallowing process has changed.

It was a complete surprise to find that the inventive use of the measurement system can overcome the disadvantages of the prior art.

Advantageously, the use of this measurement system allows rapid and easy assessment of part of or the entire swallowing process. It is particularly advantageous that the swallowing process can be assessed already during swallowing. The measurement proceeds very rapidly, thereby making the use much easier because, for example, no qualified personnel is required for evaluating X-ray images.

The new measurement system allows assessing the crucial phase of swallowing, i.e. closure of the larynx, and thus the protection of the lower respiratory tract. Advantageously, the inventive use allows easy assessment of an airway closure without exposing the patient to safety risks such as X-rays. Risks to the patient are excluded by the new measurement system.

The ease in handling allows uses of the measurement system that do not necessarily have to be performed or supervised by qualified personnel, thereby considerably reducing the costs of applications.

Another advantage is that measurements can be made immediately at any point in time. Preparatory measures, such as swallowing of contrast medium, are not required. The detected change in bioimpedance can be compared with control values. For example, it is also possible in this way to assess one's own progress.

The measurement system is preferably used in those cases where the cartilage, which at least in part forms a cavity, is coated with a mucous membrane. In a particularly preferred fashion the cavity partially or completely surrounded by cartilage is a larynx. By virtue of this embodiment it is possible to determine the entry of non-gaseous substances into the larynx and down to the vocal folds (penetration) and/or the passage of non-gaseous substances through the larynx (aspiration).

Non-gaseous substances whose passage can be determined with the measurement system can be autologous secretions, such as saliva, as well as foreign bodies. This may involve liquid materials, so that the measurement system can be used to control drinking, for example. It is also possible to determine the passage of solids, e.g. foods. Even small crumbs entering the trachea may have serious consequences if the patient is not able to immediately remove them e.g. by coughing.

Since the larynx forms the transition from the pharynx to the trachea, it is the earliest point where a developing aspiration and/or penetration can be detected. Aspiration and/or penetration can develop if the laryngeal vestibule is not completely closed by the larynx during swallowing. Consequently, the use of the measurement system in accordance with the invention therefore not only allows determination of the actual aspiration or penetration, but also enables assessing the risk of a possibly existing aspiration and/or penetration by merely observing the swallowing behavior of a patient.

In a preferred embodiment the measurement system is used to determine or diagnose aspiration. Aspiration may have life-threatening consequences so that rapid recognition thereof is of crucial importance. In this way it is possible to judge whether a patient is able to ingest food or artificial feeding is necessary. To date, such assessments frequently had to be made by feel, because the measuring methods required could be performed only by a physician and were therefore not sufficiently available in many institutions (such as nursing homes). Being non-invasive and very simple to use, the measurement system according to the invention does not require a physician for transcutaneous use. The new method allows detection of perilous transition of liquids, saliva or food into the trachea without substantial technical effort or hazardous investigations such as X-ray.

In a preferred embodiment, the measurement system is used to determine penetration. Penetration involves entry of non-gaseous substances into the upper respiratory tract. The determination of penetration can therefore be used to control the swallowing process and identify the risk of possible aspiration at an early stage.

In a particularly preferred embodiment the invention relates to the use of the measurement system, wherein the change in bioimpedance during the approach of larynx and hyoid bone is determined.

Quite surprisingly, the approach of larynx and hyoid bone results in a change in bioimpedance in the neck, which can be measured through the inventive use of the measurement system. This distinguishes the invention from previously known BI measuring methods. Thus, the decisive phase of the swallowing process, namely, closure of the airways by the approach of larynx and hyoid bone, can be assessed with the measurement system according to the invention.

Also preferred is a use of the measurement system, wherein
the voltage measuring elements are each arranged in a voltage measuring electrode, and
the elements for applying a current are each arranged in a current electrode.

This embodiment can be used transcutaneously, subcutaneously, intratracheally as well as intraluminally. The advantage of this use is that the system furnishes particularly precise data and can be adapted to the anatomy of any patient without great effort.

More specifically, there are two possible procedures of measuring the BI. In the 2-electrode method, the voltage is measured directly via the current electrodes, the voltage drop being measured simultaneously via the electrode-skin contact. The voltage drop is caused by the current flow generated in the patient via the current electrodes. This resistance varies with time and therefore leads to measurement errors. Such an undesirable effect can be avoided by using the 4-electrode method where the voltage is measured via additional electrodes. Because the current flowing through the voltage electrodes is negligible, there is no interfering time-varying voltage drop as a result of electrode-skin contact. The preferred embodiment of the measurement system supports both methods.

Surprisingly, the measurement system can also be used to determine the approach of larynx and hyoid bone and the aspiration and/or penetration at the same time. Aspiration can occur if the larynx is not properly closed during swallowing. For this reason, swallowing disorders, such as dysphagia, and aspiration or penetration can be mutually dependent. Simultaneous assessment may therefore be advantageous. Allowing detection of both sufficient closure of the larynx during the swallowing process and passage of non-gaseous substances through the larynx, this use is particularly suitable for the assessment of swallowing behavior and/or the diagnosis, therapy or prevention of aspiration, penetration and/or dysphagia. The measurement system is superior to the known systems because dysphagia as well as aspiration or penetration can be detected in a particularly uncomplicated way. Thus, the measurement system of the invention allows detection of a predestination for aspiration as well as the aspiration itself.

It may be advantageous for this use of the measurement system to have two power sources which in this case must differ in their frequency range so that the two BI measuring methods do not influence each other. Preferably, one bioimpedance measurement is performed at 50 kHz and the other bioimpedance measurement is preferably carried out at 100 kHz.

The established correlation between BI change and larynx-hyoid distance allows automated assessment of the swallowing process, which can be used in visual representation or to control other devices (screen display, stimulators). This opens up completely new possibilities for the diagnosis, therapy and prevention of swallowing disorders.

For use of the measurement system the current electrodes in a particularly preferred embodiment are arranged on both sides on the sternocleidomastoid muscle at the level of the lower jaw and/or on the thyroid cartilage at the level of or below or above the vocal cord plane.

These positions were found to be particularly advantageous because the measurement values obtained were particularly accurate.

The arrangement of the current electrodes on both sides on the sternocleidomastoid muscle at the level of the lower jaw is particularly suitable for use of the measurement system for determining the closure of the airways during the swallowing process. Surprisingly, these positions are also particularly advantageous for use of the measurement system for simultaneous assessment of the closure of the airways during the swallowing process and determination of aspiration and/or penetration.

Arrangement of the current electrodes on the thyroid cartilage at the level of or below the vocal cord plane was found to be advantageous for use of the measurement system for determining aspiration. Penetration could be detected particularly well when the current electrodes were arranged on the thyroid cartilage above the vocal cord plane.

It is also particularly preferred to use the measurement system in such a way that the voltage measuring electrodes are arranged on both sides between the hyoid bone and thyroid cartilage in front of the sternocleidomastoid muscle and/or on the thyroid cartilage at the level of or below or above the vocal cord plane.

It was found that the change in bioimpedance during the approach of larynx and posterior hyoid bone can be detected particularly well when the voltage measuring electrodes are arranged on both sides between the hyoid bone and thyroid cartilage in front of the sternocleidomastoid muscle.

The arrangement of the voltage measuring electrodes on the thyroid cartilage at the level of or below the vocal cord plane has proven particularly suitable for determining the risk of aspiration. By arranging the voltage measuring electrodes on the thyroid cartilage above the vocal cord plane, particularly advantageous measurement of the change in bioimpedance during penetration is possible.

In another preferred embodiment the invention relates to the use of the measurement system, wherein one voltage measuring element and one element for applying a current are arranged together in a single electrode. This measurement system can be used transcutaneously, subcutaneously, intratracheally or intraluminally. This embodiment is advantageous because the use thereof is quite agreeable for a patient and is not considered annoying. Owing to its compact design, the system is suitable for mobile use, can be easily transported and is particularly easy to use by patients themselves.

This embodiment is also advantageous in applications where permanent detection is required, such as detection of saliva passage. Reducing the number of electrodes required is particularly suitable for subcutaneous, intratracheal or intraluminal application, because the measurement system can be applied gently and is not felt as painful or considered annoying.

In another particularly preferred embodiment the electrodes, each comprising a voltage measuring element and an element for applying a current, are arranged on both sides in front of the sternocleidomastoid muscle between the hyoid bone and thyroid cartilage or on both sides on the thyroid cartilage at the level of or below or above the vocal cord plane. This arrangement is particularly well suited to detect passage or penetration of non-gaseous substances through the larynx. Surprisingly, this arrangement also allows detection of extremely small volumes, so that the passage of small amounts of liquid or crumbs can be detected.

In particularly preferred embodiments, aspiration is determined using one of the following electrode arrangements:
Four electrodes (two current electrodes and two voltage measurement electrodes) are attached transcutaneously, essentially along a line, on both sides of the thyroid cartilage at the level of or below the vocal cord plane. For particularly precise measurement, the two outer electrodes are preferably current electrodes.
Two electrodes, each comprising a voltage measurement element and an element for applying a current, are attached transcutaneously on both sides of the thyroid cartilage at the level of or below the vocal cord plane.
Two current electrodes and two voltage measurement electrodes are arranged subcutaneously on, in or through the thyroid cartilage at the level of or below the vocal cord plane, the two outer electrodes preferably being current electrodes.
Two electrodes, each comprising a voltage measurement element and an element for applying a current, are arranged subcutaneously on, in or through the thyroid cartilage at the level of or below the vocal cord plane.
Two current electrodes are located on or in a tracheal cannula, while two voltage measurement electrodes are arranged transcutaneously on the thyroid cartilage at the level of or below the vocal cord plane or subcutaneously on, in or through the thyroid cartilage.
Two electrodes, each comprising a voltage measurement element and an element for applying a current, are located on a tracheal cannula.

In particularly preferred embodiments, penetration is determined using one of the following electrode arrangements:
Four electrodes (two current electrodes and two voltage measurement electrodes) are attached transcutaneously, essentially along a line, on both sides of the thyroid cartilage above the vocal cord plane. For particularly precise measurement, the two outer electrodes are preferably current electrodes.
Two electrodes, each comprising a voltage measurement element and an element for applying a current, are attached transcutaneously on both sides of the thyroid cartilage above the vocal cord plane.
Two current electrodes and two voltage measurement electrodes are arranged subcutaneously on, in or through the thyroid cartilage above the vocal cord plane, the two outer electrodes preferably being current electrodes.
Two electrodes, each comprising a voltage measurement element and an element for applying a current, are arranged subcutaneously on, in or through the thyroid cartilage above the vocal cord plane.
Two current electrodes are located on or in a tracheal cannula, while two voltage measurement electrodes are arranged transcutaneously on the thyroid cartilage above the vocal cord plane or subcutaneously on, in or through the thyroid cartilage.
Two electrodes, each comprising a voltage measurement element and an element for applying a current, are located on a tracheal cannula.
In particular, cannulas/tubes and sensors should be used if already present. Items already present in the neck can thus be used with advantage. Applications are possible even when measuring via the cannula alone (e.g. measurement of aspiration with cannula or tube in position; flow passing despite cannula).

In particularly preferred embodiments the swallowing process, preferably the approach of larynx and hyoid bone, is assessed using one of the following electrode arrangements:
The current electrodes are attached transcutaneously on both sides on the sternocleidomastoid muscle at the level of the mandibular angle, while the voltage measurement electrodes are arranged on both sides between the hyoid bone and thyroid cartilage in front of the sternocleidomastoid muscle.
The electrodes, each comprising a voltage measurement element and an element for applying a current, are transcutaneously attached laterally on both sides of the neck in front of the sternocleidomastoid muscle between hyoid bone and thyroid cartilage.
The current electrodes are attached subcutaneously on both sides on the sternocleidomastoid muscle at the level of the mandibular angle, while the voltage measurement electrodes are arranged subcutaneously at the level of the epiglottic vallecula.
The electrodes, each comprising a voltage measurement element and an element for applying a current, are attached subcutaneously at the level of the epiglottic vallecula.
Two current electrodes are arranged on a sensor located in the pharynx, while the two voltage measurement electrodes are attached transcutaneously between hyoid bone and thyroid cartilage or subcutaneously at the level of the epiglottic vallecula.
Each electrode comprises a voltage measurement element and an element for applying a current, and one electrode is arranged on a sensor located in the pharynx, while the other electrode is attached subcutaneously between hyoid bone and thyroid cartilage or subcutaneously at the level of the epiglottic vallecula.

In particularly preferred embodiments the swallowing process, preferably the approach of larynx and hyoid bone, is assessed, while the incidence of aspiration and/or penetration is determined at the same time, using one of the following electrode arrangements:
When using a measurement configuration with only one power source, two transfer bioimpedances are measured with respect to this power source.
The current electrodes are attached transcutaneously on both sides on the sternocleidomastoid muscle at the level of the mandibular angle, while two voltage measurement electrodes are attached transcutaneously on both sides between hyoid bone and thyroid cartilage in front of the sternocleidomastoid muscle and two additional voltage measurement electrodes are attached transcutaneously on both sides on the thyroid cartilage at the level of or below or above the vocal cord plane.
The current electrodes are attached subcutaneously on both sides on the sternocleidomastoid muscle at the level of the mandibular angle, while two voltage measurement electrodes are attached subcutaneously at the level of the epiglottic vallecula and two additional voltage measurement electrodes are attached subcutaneously at the level of or below or above the vocal cord plane.
The current electrodes are located on or in a pharyngeal sensor or on or in a tracheal cannula, while two voltage measurement electrodes are attached subcutaneously at the level of the epiglottic vallecula and two additional voltage measurement electrodes are attached subcutaneously at the level of or below or above the vocal cord plane.
It is also possible to combine all the above-mentioned arrays for assessing the swallowing process with all the above-mentioned arrays for determining aspiration or penetration, in which event one bioimpedance measurement is preferably carried out at 50 kHz and the other bioimpedance measurement preferably at 100 kHz. Advantageously, this prevents mutual influence of the two BI measurement procedures, which would falsify the measurement result.

The respective terms have a generally accepted meaning in the relevant field of the art, so that a person skilled in the art will be able to implement the above-mentioned uses of the measurement system.

The positions specified herein are particularly suited to obtain precise measurement values because large amplitudes can be achieved. Furthermore, no false-positive or false-negative results are generated with the measurement arrays specified above. Also, these positions are well-suited for the applications mentioned above because the electrodes can be arranged on the appropriate spots in an uncomplicated manner and do not slip as a result of the swallowing process or breathing movements. A person skilled in the art will interpret the technical teaching of the present invention in such a way that the anatomy of a patient may require slight variations in the positions of the electrodes in accordance with gender, height, weight and age. A person skilled in the art will be able, depending on the anatomical preconditions of the individual patient, to arrange the electrodes in such a way that optimum measurement is possible.

In another preferred embodiment the invention relates to the use of the measurement system, which system additionally includes neuromuscular stimulators. Thus, the measurement system is not only usable in the determination of swallowing disorders, dysphagia, aspiration and/or penetration, but is also capable of initiating appropriate counter measures. When aspiration and/or penetration occur, swallowing, coughing or clearing the throat can be initiated by appropriate stimulation, for example. The data obtained from the measurements can be used to control different types of stimuli, e.g. electrically, mechanically, chemically (e.g. citric acid), thermally or by means of vibration, to make the process of swallowing safer.

It is possible, for example, to stimulate the digastric muscle, geniohyoid muscle and/or thyrohyoid muscle. In addition, a cough can be triggered by stimulating the superior laryngeal nerve, the vagus nerve and/or stretch receptors of the trachea. To this end, the stimulation pulses are generated using a multichannel stimulator for neuromuscular stimulation.

The method represents a significant advance in the diagnosis of life-threatening aspiration. When detecting aspiration, it is possible to identify undiscovered aspiration (“silent aspiration”) which is especially dangerous to a patient due to the absence of defensive reactions (coughing, clearing the throat) protecting the patient. Coughing can be induced by triggered stimulation (electrical, chemical, mechanical, etc.). The invention not only enables easier detection of aspiration, but also allows triggering a defensive reaction without significant time delay, so that the risk of developing secondary diseases is notably minimized.

In a preferred fashion the measurement system is used in the form of a neural prosthesis for subcutaneous, intratracheal or intraluminal application. This embodiment is particularly suitable for continuous use, allowing e.g. permanent control of the swallowing process by the patient. Quite surprisingly, it was possible to design the measurement system of the invention in such a way that successful implantation in a patient can be made without any complications or impairment of the patient.

The use of a neural prosthesis for the treatment of swallowing disorders is of enormous clinical significance, providing a new, forward-looking perspective in the rehabilitation of swallowing disorders. To date, there are only few valid therapeutical options for the treatment of neurogenic dysphagia. In most cases of patients suffering from severe dysphagia, diagnosis is followed by application of a tracheal cannula to minimize aspiration and use of a PEG (percutaneous endoscopic gastrostomy) to maintain food intake. In addition, an intensive, lengthy rehabilitation therapy with uncertain outcome is necessary. This process often takes years to complete. During this period, the patient is dependent on 24-hour care, rehabilitation and medical aid. This represents a significant burden to patients, relatives and cost centers. By virtue of the supporting measures of controlled stimulation as part of a neural prosthesis, it is possible to change and shorten this course, which means significant reduction of the impairment and improvement of the quality of life for the patients and substantial savings for cost centers.

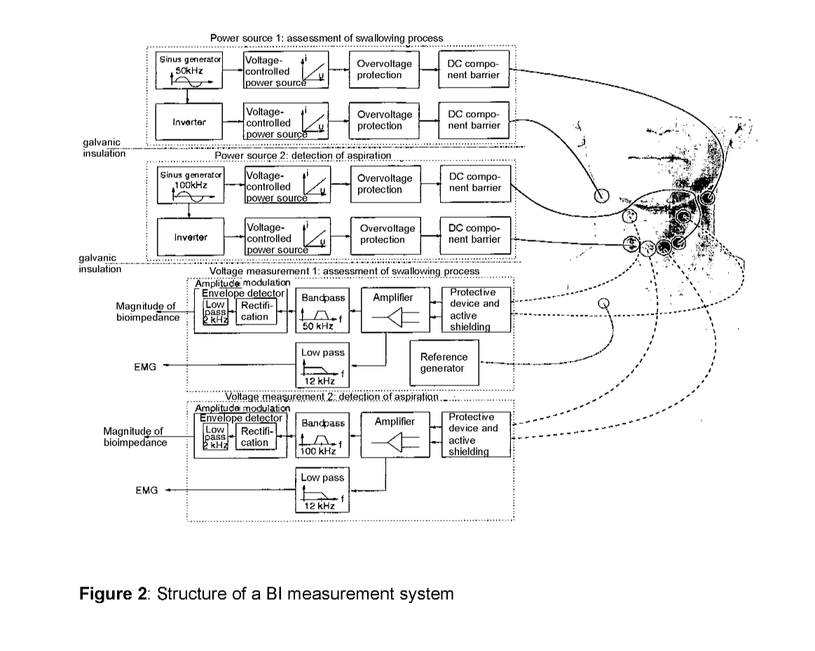

A preferred embodiment of a measurement system in accordance with the invention is shown in FIG. 2. To protect the measurement amplifier against the high voltage of the stimulation pulses, the input of the amplifier is protected. Such protection can be achieved by using stimulation pulse-triggered switches, diodes or the like. It is preferred to use a combination of resistors and diodes. In a preferred fashion, resistors are arranged at the input of the measurement amplifier, followed by diodes capable of draining off the high voltage drops on the voltage electrodes. In addition, such protection prevents dangerous leakage currents to the patient. The output of the power source is protected by diodes capable of draining off the high voltage drops on the current electrodes.

In addition, the system was designed for use with neuromuscular stimulators. Such stimulation systems are being used in the therapy of swallowing disorders or in neuroprosthetic assistance for dysphagia patients.

In another preferred embodiment the invention relates to the use of the measurement system for producing a means for the assessment of the swallowing process, supporting the therapy of swallowing disorders and/or diagnosing changes in the swallowing process.

In another preferred embodiment the invention relates to the use of the measurement system for producing a means for the diagnosis, therapy and/or prevention of aspiration, penetration and/or dysphagia.

It is preferred to use a measurement system at a frequency of 25 kHz to 200 kHz, preferably 50 kHz or 100 kHz.

It is also preferred to use a bandpass filter. In this way it is possible to remove disturbing artefacts and isolate the measuring frequencies.

The bandpass filter is preferably inserted downstream of the measurement amplifier. Disturbing artefacts on the voltage measuring elements, which may be caused by muscle action potentials and cable movement, can be easily removed by means of the bandpass, because the changes in BI are included in the amplitude-modulated voltage within a narrow frequency range around the selected measurement frequency.

Furthermore, this measuring method is more robust to external interference and has higher time resolution than, for example, electrotomography and multi-channel electroglottography.

Also preferred is the use of the measurement system for EMG measurement, in which event a low-pass filter with preferably 12 kHz is used. The low-pass filter allows isolation of the measurement frequency and thus removal of disturbing artefacts. The low-pass filter is preferably inserted downstream of the measurement amplifier. The BI measuring voltages on the voltage measuring elements, which may be caused by the power source, can be easily removed by means of the low pass, because the muscle action potentials are included in the selected measurement frequency.

The EMG allows measurement of existing muscle activity during the swallow and thus provides further information for assessing the swallowing process. Owing to the simultaneous measurement on the voltage electrodes, use of additional electrodes is not required.

Particularly advantageous is a use wherein at least one differential power source is used, which symmetrically controls the floating load, minimizes and preferably suppresses common-mode interference, allows essentially no DC component, and is robust to grounding the load to earth. This embodiment is advantageous because it largely prevents DC currents during measurement and measurement errors as a result of patient contact and is robust to grounding the load to earth. It is particularly preferred to use a power source in accordance with DE 601 25 601 T2. The DE 601 25 601 T2 is hereby incorporated in the disclosure of the present invention.

It is also preferred to use a direct current barrier to the patient. The DC barrier is preferably placed on both outputs of the power source. The DC barrier is preferably arranged downstream of the diodes used for surge protection. In preferred embodiments, capacitors, preferably Y1 capacitors, are used as DC barrier. It is also preferred to use complete galvanic insulation of each differential power source.

The circuit-technical implementation of the BI measurement is simplified because only the changing magnitude of the BI needs to be determined. The course thereof can be obtained from the envelope of the measured voltage. It is also preferred to use a measurement system wherein the magnitude of the bioimpedance is determined via amplitude modulation, preferably using an envelope detector.

By controlling two current flows via both current electrodes, it is possible to avoid some of the problems of the prior art. While touching the patient during measurement likewise results in current flow through the therapist, this flow is compensated by readjusting the current flow, thereby eliminating one major source of error.

To avoid a voltage drop of the changing electrode-skin impedance and the associated measurement error, it is preferred to use the 4-electrode method for transcutaneous measurements.

In another preferred embodiment the invention relates to the use of the measurement system, wherein the change in bioimpedance is displayed visually, preferably on a monitor. The data obtained from the measured values can be used for visual representation to assist the patient in performing exercises as part of a therapy. This embodiment is advantageous because it allows monitoring which need not be supervised by a physician or specialist personnel.

In another preferred embodiment, a reference generator (“right leg drive”) can be used. This circuit ensures that capacitively coupled common-mode interference is prevented or at least reduced.

Thus, alternatively, it is possible with advantage to perform the measurement without a reference electrode when instead activating an active circuit for common-mode reduction at the inputs of the voltage measuring circuit. The principle of “active shielding” is used to reduce the capacity of the measurement cable, involving active generation of a shield voltage which is applied to the cable shield of the voltage measurement elements.

Accordingly, the invention relates to a measurement system for use in assessing a swallowing process, said system comprising:
two elements for applying a current,
the current being applied to the neck region, and a change in bioimpedance being detected
by two voltage measuring elements for
(i) detecting a closure of the airways during the swallowing process and/or
by two voltage measuring elements for
(ii) detecting the passage of non-gaseous substances through a cavity partially or completely surrounded by cartilage.

The above explanations for the use of the system also apply to the system for use in assessing a swallowing process.

Other advantages of the invention are as follows:
Easy handling
Low risk to the patient
Reliable assessment
Measurement is possible over 24 hours and 7 days a week.
BI measurement allows unattended monitoring and recording of the swallowing process and/or aspiration
The system can be used for therapy monitoring.

### EXAMPLES

### Example 1

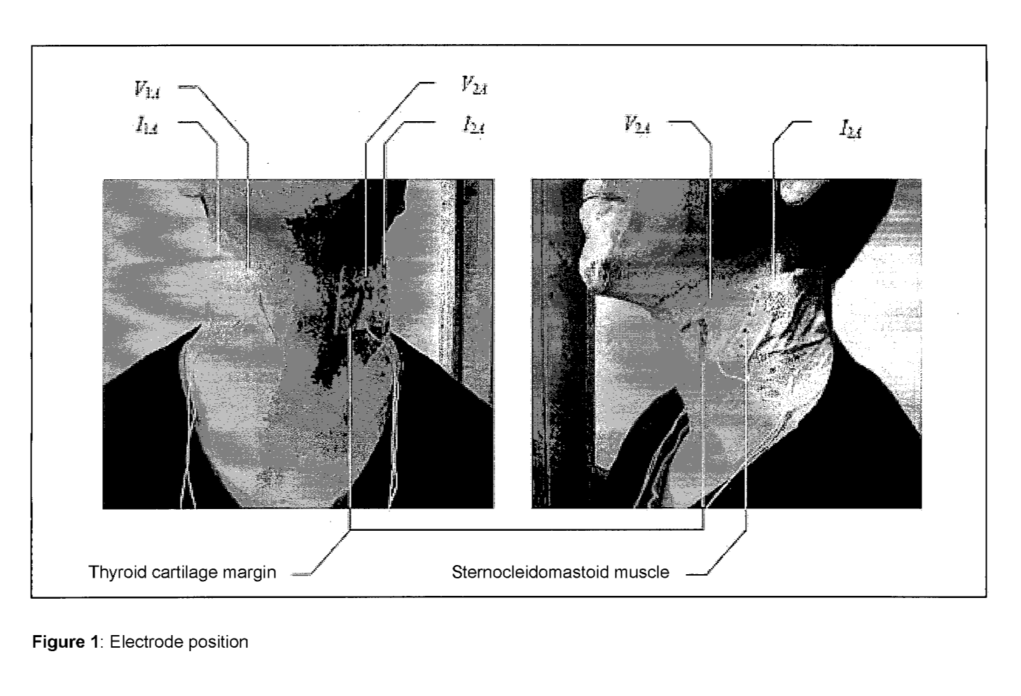

In a pilot study, two patients with normal swallowing who had to undergo an X-ray examination with contrast medium for diagnosing their malignant disease were simultaneously subjected to a BI measurement wherein the change in BI magnitude was measured using the 4-electrode method. The voltage measurement electrodes were adhered on both sides at the level of the thyroid cartilage posterior horn. Current was fed on both sides through electrodes on the sternocleidomastoid muscle. FIG. 1 exemplifies the electrode positions/points of measurement.

The amplitude of the sinusoidal current was 0.25 mA, and the frequency was 50 kHz. The four-electrode method with separate transcutaneous current electrodes and voltage electrodes was chosen in order to avoid voltage drop of the changing electrode-skin impedance and associated measurement errors. In the event of an implanted measurement system, it is also possible to use a two-electrode measurement array (where current electrodes and measurement electrodes are identical).

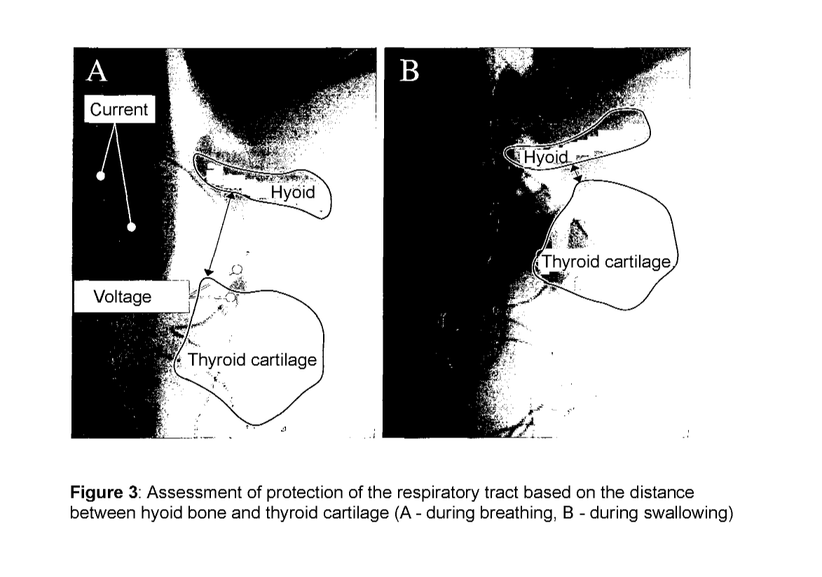

The hypothesis to be tested in the pilot study was whether assessment of a laryngeal closure could be made by means of a bioimpedance measurement. The distance between the posterior thyroid cartilage (larynx) and the hyoid bone (hyoid) was used as a reference for laryngeal closure (see FIG. 3).

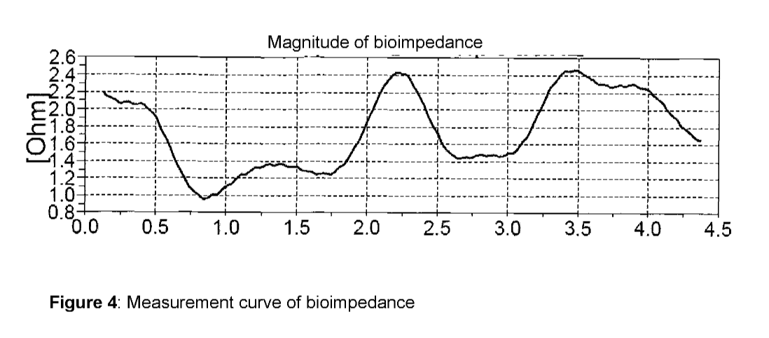

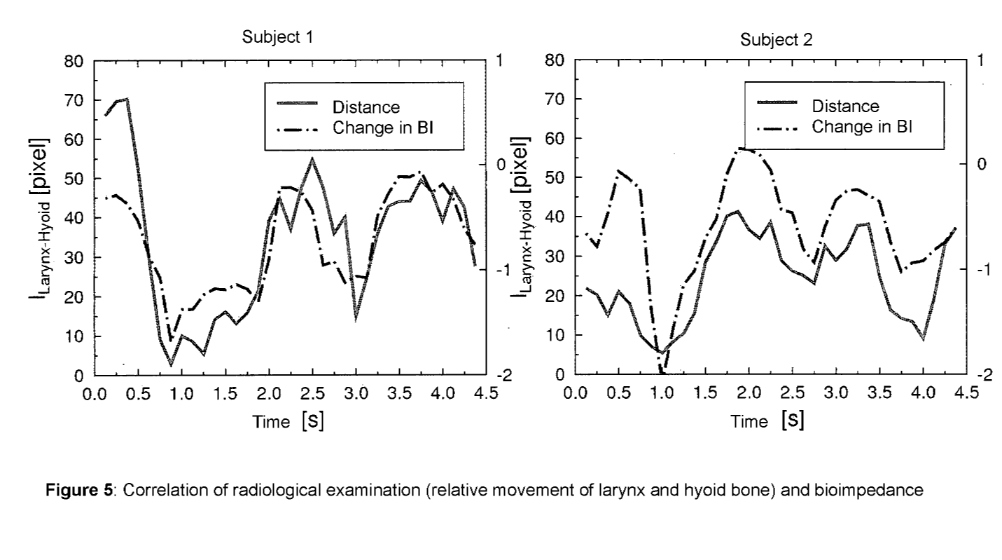

FIG. 4 shows the change in bioimpedance magnitude versus time. Compared to the simultaneous radiological examination, it was found that the bioimpedance magnitude decreases when the anatomical structures approach each other (see FIG. 5). The amplitude of the BI correlates with the degree of airway closure. Linear regressions of BI change and distance yielded the correlation coefficient R_{Subject 1}=0.65 and R_{Subject 2} =0.51.

### Example 2

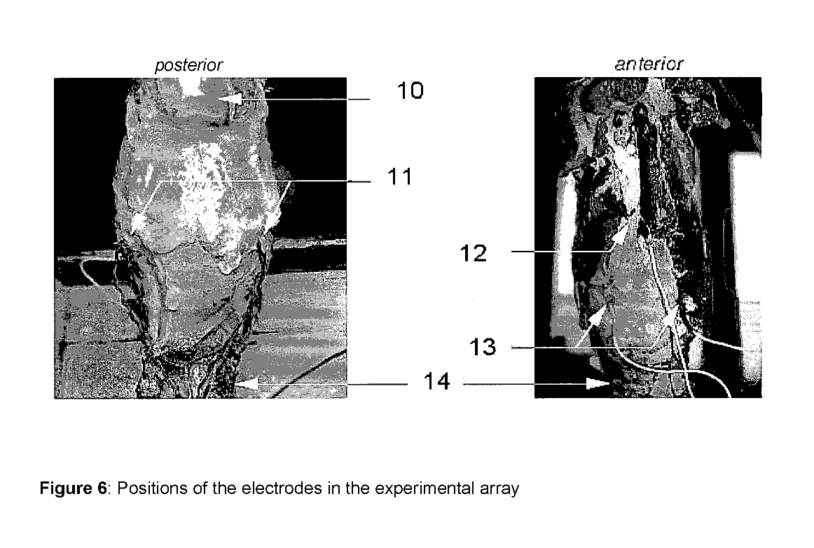

Using the 4-electrode method, the change in BI magnitude was measured on a bovine larynx. The voltage measurement electrodes were attached to both sides at the level of the arytenoids in the muscles. Current was fed on both sides through electrodes inserted in the thyroid cartilage (no passage of the electrodes into the cavity). FIG. 6 exemplifies the electrode positions and points of measurement.

The four-electrode method (separate electrodes for current feeding and voltage measurement) was used for measurement, but it is also possible to use a two-electrode measurement array (where current electrodes and measurement electrodes are identical).

The amplitude of the sinusoidal current was 0.25 mA, and the frequency was 50 kHz.

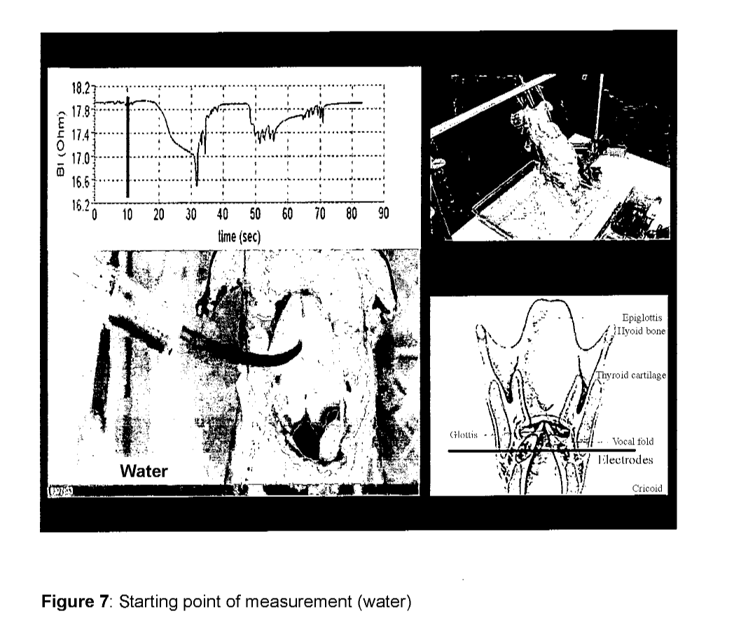

The hypothesis to be tested was whether passage of non-gaseous substances through the larynx would result in a change in bioimpedance. The results of an experimental series of measurements are shown in FIGS. 7 to 12. It was found that the impedance changes depending on the selected fluids. A specific minimum can be observed for each individual fluid, which depends on the ionic charge (conductivity) of the fluid. The measurement curve returns to the starting point after passage of the fluids, and there are no summation effects.

FIGS. 7 through 11 show the passage of fluids through the larynx. Shown are the measurement curve (top left), the clamped bovine larynx (top right), the video image with a view inside the larynx and the names of the fluids (bottom left), and a schematic representation of the measuring point level (bottom right).

FIG. 7: Identifies the starting point.

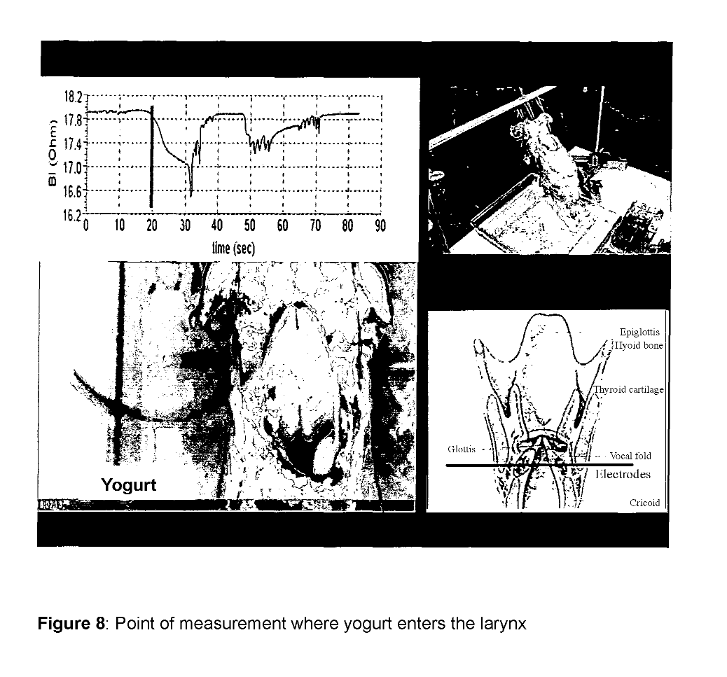

FIG. 8: Yogurt enters the larynx.

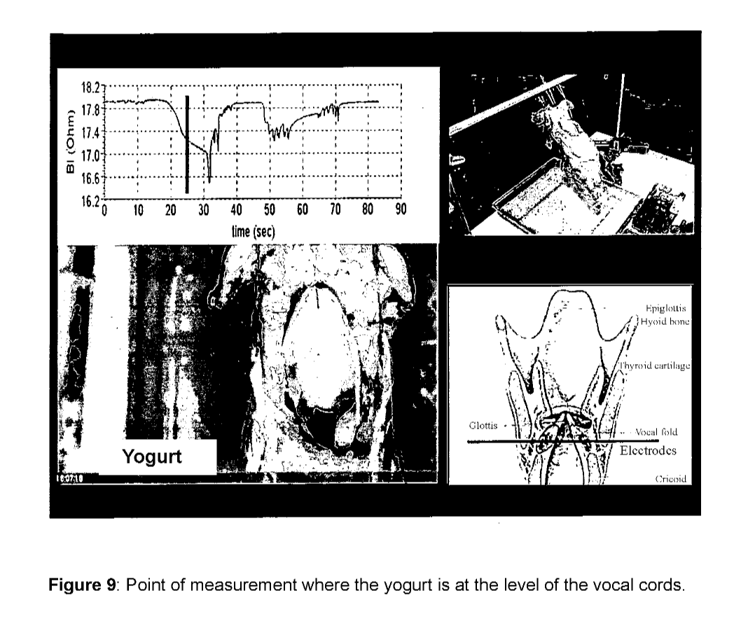

FIG. 9: Yogurt is located at the level of the vocal cords, just above the measurement points, a marked change of the measurement curve is seen.

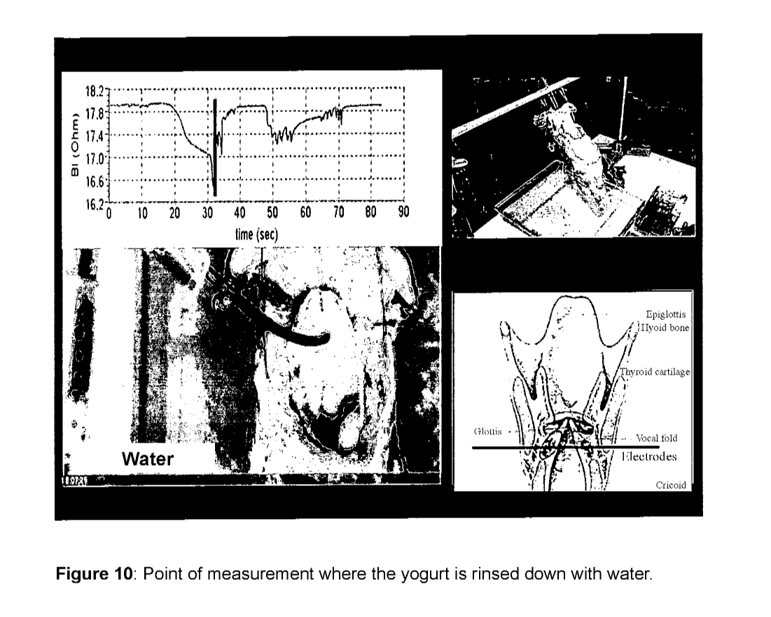

FIG. 10: The yogurt is rinsed down with water, the measurement curve reaches its minimum upon passage beyond the measurement points.

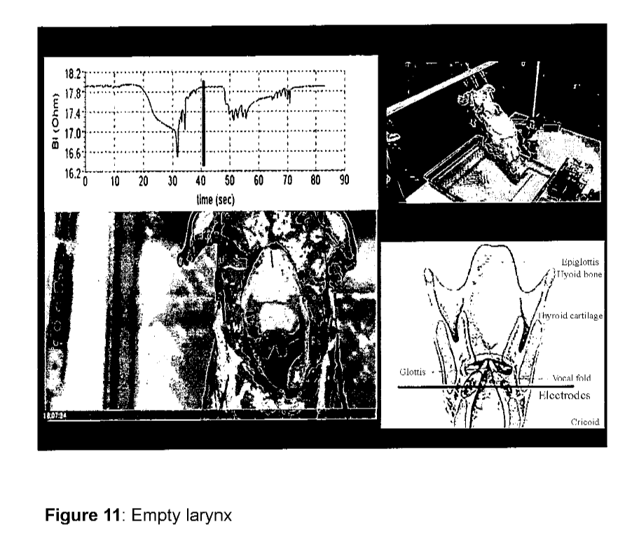

FIG. 11: The measurement curve returns to the starting point as soon as the larynx is empty.

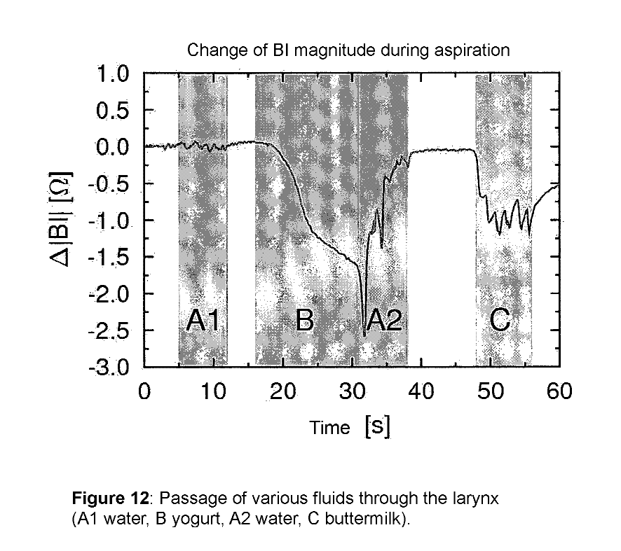

FIG. 12: Shows the passage of various fluids through the larynx (A1 Water, B yogurt, A2 water, C buttermilk). In each case the minimum describes the passage of fluid beyond the point of measurement.

### REFERENCE NUMERALS

10 Laryngeal vestibule
11 Current electrodes
12 Reference electrode
13 Voltage electrodes
14 Trachea

1. A measurement system for assessment of a swallowing process, said system comprising
two elements for applying a current adapted for applying the current to a neck region,
(a) two voltage measuring elements for
(i) detecting a closure of the airways during the a swallowing process and/or
(b) two voltage measuring elements for
(ii) detecting a passage of non-gaseous substances through a cavity partially or completely surrounded by cartilage, wherein (a) and/or (b) detect a change in bioimpedance.

2. A measurement system as claimed in claim 1, wherein the cavity is a larynx.

3. (canceled)

4. (canceled)

5. A measurement system as claimed in claim 1, wherein
the voltage measuring elements of (a) and/or (b) are each arranged in a voltage measuring electrode, and
the elements for applying a current are each arranged in a current electrode.

6. A measurement system as claimed in claim 1, wherein one voltage measuring element and one element for applying a current are arranged together in a single electrode.

7. (canceled)

8. (canceled)

9. (canceled)

10. (canceled)

11. A measurement system as claimed in claim 1, wherein said system provides a frequency of from 25 kHz to 200 kHz, preferably 50 kHz or 100 kHz.

12. A measurement system as claimed in claim 1, wherein two power sources differing in their frequency range are provided.

13. A measurement system as claimed in claim 1, wherein a bandpass filter is provided to eliminate disturbing artefacts and isolate the measuring frequency.

14. A measurement system as claimed in claim 1, wherein a differential power source is provided, which symmetrically controls a floating load, minimizes or suppresses common-mode interference, allows essentially no DC component, and is robust to grounding the load to earth.

15. (canceled)

16. (canceled)

17. A method for assessing a swallowing process of a subject comprising:
providing the measurement system according to claim 1,
applying the current to the neck region of said subject via said two elements and detecting the change in bioimpedance by
detecting the closure of the airways during the swallowing process via said two voltage measuring elements in (a) and/or
by detecting the passage of non gaseous substances through said cavity partially or completely surrounded by cartilage via said two voltage measuring elements in (b).

18. The method of claim 17, wherein said cavity is larynx.

19. The method of claim 17, wherein aspiration and/or penetration is detected.

20. The method of claim 19, wherein the change in bioimpedance is determined during the approach of the larynx and hyoid bone.

21. The method of claim 17, wherein the electrodes are arranged on both sides of a sternocleidomastoid muscle at a level of the lower jaw and/or on a thyroid cartilage at a level of or below or above a vocal cord plane.

22. The method of claim 17, wherein the voltage measuring elements are arranged on both sides between hyoid bone and thyroid cartilage in front of the sternocleidomastoid muscle and/or on the thyroid cartilage at the level of or below or above a vocal cord plane.

23. The method of claim 17, wherein one voltage measuring element and one element for applying a current are arranged together in a single electrode and wherein the electrodes are arranged on both sides in front of a sternocleidomastoid muscle between hyoid bone and thyroid cartilage or on both sides on the thyroid cartilage at a level of or below or above a vocal cord plane.

24. A method for diagnosing a swallowing disorder in a subject comprising providing the measurement system according to claim 1,
applying the current to the neck region of said subject via said two elements and detecting the change in bioimpedance by
detecting said closure of the airways during the swallowing process via said two voltage measuring elements in (a) and/or
by detecting the passage of non gaseous substances through said cavity partially or completely surrounded by cartilage via said two voltage measuring elements in (b) and thereby diagnosing said swallowing disorder in said subject.

25. The method of claim 17, wherein said assessment of the swallowing process, supports a therapy of swallowing disorders and/or allows for diagnosing changes in the swallowing process.

26. A method for a diagnosing, treating and/or preventing aspiration, penetration and/or dysphagia in a subject comprising
providing the measurement system according to claim 1,
applying the current to the neck region of said subject via said two elements and detecting the change in bioimpedance by
detecting a closure of the airways during the swallowing process via said two voltage measuring elements in (a) and/or
by detecting the passage of non gaseous substances through said cavity partially or completely surrounded by cartilage via said two voltage measuring elements in (b) and further diagnosing, treating and/or preventing said aspiration, penetration and/or dysphagia in said subject.
